(19) United States
(12) Patent Application Publication
(10) Pub. No.: US 2010/0256661 A1
(43) Pub. Date: Oct. 7, 2010

(54) APPARATUS AND METHOD FOR ENABLING PERFORATING VEIN ABLATION

(76) Inventor: Zeev Brandeis, Rosh Haayin (IL)

Correspondence Address: MARTIN D. MOYNIHAN d/b/a PRTSI, INC., P.O. BOX 16446, ARLINGTON, VA, 22215, US

(21) Appl. No.: 12/418,797

(22) Filed: Apr. 6, 2009

(51) Int. Cl. A61B 17/22

(52) U.S. Cl. 606/159

(57) ABSTRACT

A device, apparatus and method are provided to implement minimally invasive perforating vein treatment, the apparatus including a vein ablation blocking element, to be deployed downstream from the vein ablation apparatus, before a deep vein junction, to prevent passage to a downstream deep vein; and an anchoring mechanism, to anchor the vein ablation apparatus into the perforating vein wall(s), to prevent movement of the vein ablation apparatus after deployment.

### CROSS REFERENCE TO RELATED APPLICATIONS

This application claims priority from U.S. Provisional Patent Application No. 60/042,802, filed Apr. 7, 2008, entitled “APPARATUS AND METHOD FOR ENABLING PERFORATING VEIN ABLATION”, which is incorporated in its entirety herein by reference.

### FIELD OF THE INVENTION

The present invention relates to methods and devices useful in treating veins. Specifically, embodiments of the present invention relate to systems, methods and apparatuses that enable minimally invasive vein ablation.

### BACKGROUND OF THE INVENTION

Perforator insufficiency occurs where blood flow is flowing from deep veins via the perforating veins to the saphenous veins, tributary veins, peroneal veins etc. This insufficiency is caused by perforant vein insufficiency or failure, for example, due to blood pressure in the deep vein being too great relative to the blood pressure in the perforant vein. Such a situation may cause significant vein discomforts and hazards, and yet is currently substantially non treatable owing to the close proximity of the perforating veins to the inner deep veins.

It would be advantageous to have a treatment system or method that could enable safe perforant vein treatment.

### SUMMARY OF THE INVENTION

There is provided, in accordance with an embodiment of the present invention, an apparatus, system, and method for minimally invasive perforating vein ablation.

According to some embodiments, a vein ablation apparatus for implementing perforating vein treatment may comprise a vein ablation blocking element, to be deployed downstream from the vein ablation apparatus, before a deep vein junction, to prevent passage to a downstream deep vein; and an anchoring mechanism, to anchor the vein ablation apparatus into the perforating vein wall(s), to prevent movement of the vein ablation apparatus after deployment.

In some embodiments the blocking element is a controllable mesh element designed to block off an area of greatest diameter in a target vein.

In some embodiments the anchoring mechanism includes hooks designed to catch each other following the constriction of the anchoring apparatus, thereby locking the vein walls together.

In some embodiments the anchoring mechanism is pre-configured to deploy at an angle appropriate for anchoring into a vein wall.

In some embodiments the anchoring mechanism may include one or more of hooks, anchors, pins, and latches.

In some embodiments the anchoring mechanism is attached to the distal and/or proximal ends of the vein ablation apparatus.

In some embodiments the anchoring mechanism includes an initial positioning fixing mechanism to enable initial sealing of the perforating vein ablation apparatus position when initially deployed.

In some embodiments the vein ablation apparatus includes one or more expandable threads coupled to surgical suture.

In some embodiments the vein ablation apparatus includes an external vein locking mechanism that is deployable from an external position adjacent to a target vein, to help anchor the anchoring mechanism to a vein wall.

According to some embodiments, a method for enabling perforating vein treatment is provided, including entering a netting device into a target vessel using an introducing catheter; when the netting device is in position, expanding a balloon to expand the netting towards a target vessel wall; releasing one or more anchors, thereby forcing the anchors around the netting, until the anchors penetrate the vessel walls, thereby anchoring the device to the vessel walls; and deflating the balloon, thereby shrinking the ablation apparatus, and causing the anchored vein walls to be collapsed.

In some embodiments the method includes removing the netting together with the balloon.

In some embodiments the method includes using a syringe driver to guide the ablation device directly from outside the limb.

In some embodiments the method includes using a syringe driver syringe with a pre-loaded needle to deploy the vein ablation device.

In some embodiments the method includes retracting the syringe plunger to aspirate blood or debris before, during or following deployment of the ablation device.

In some embodiments the method includes retracting the syringe plunger to manipulate the positioning of the vein ablation device.

According to some embodiments, a perforant vein ablation device is provided, that includes a netting based device that surrounds an expandable balloon, the device being deliverable via a catheter; one or more anchors for anchoring the device in a target vein, the anchors being positionable at the base of the ablation device, and the anchors being deployable by releasing them beyond the base.

In come embodiments the anchors are constructed from one or more materials selected from the group consisting of Nitinol, stainless steel, or other memory alloys or metals.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIGS. 1A-1C are schematic diagrams illustrating various perspectives and/or types of deployments of a perforating vein ablation apparatus in a vein, according to some embodiments;

FIG. 2 is a schematic diagram illustrating one or more deployable elements in an ablation device, according to some embodiments;

FIG. 3 is a schematic diagram illustrating the deployment of an ablation device in a perforating vein, according to some embodiments;

FIGS. 4A and 4B are schematic illustrations showing deployment mechanisms for controlling a vein ablation procedure, according to some embodiments;

FIG. 5 is a schematic diagram illustrating the deployment of an ablation device in a perforating vein, according to some embodiments; and

FIGS. 6A and 6B are schematic diagrams illustrating respectively the deployment of an ablation device in a perforating vein, and an ablation apparatus, according to some embodiments.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The term “perforating vein” as used herein may encompass other veins, including deep veins, arteries, and other suitable treatment areas.

Embodiments of the present invention enable perforating vein ablation in a minimally invasive way. According to some embodiments of the present invention, a vein ablation device includes a vein occluding mechanism that is accurately and permanently positioned in the target vein using a syringe driver guided by ultrasound. The term “vein ablation”, as used herein, may also refer to vein destruction, vein elimination, vein occlusion, vein sclerosing, vein restriction or other forms of vein treatments.

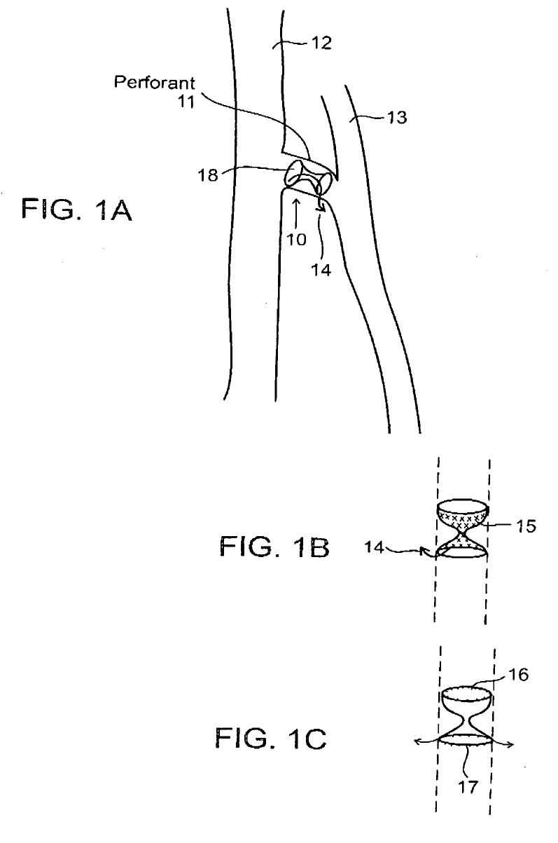

Reference is now made to FIGS. 1A-1C which are schematic diagrams illustrating various instances of the deployment of a perforating vein ablation apparatus 10 in a vein, according to some embodiments. As can be seen in FIG. 1A, perforating vein ablation apparatus 10 may be deployed and fixed at a specific selected location in a perforating, perforant or collateral vein 11. The perforating vein ablation apparatus 10 may include a blocking or occluding element 18, for example, an expandable mesh (e.g., stainless steel or other alloys, medical polymer, or Nitinol based etc.) or cap (e.g., PTFE). The mesh or cap may, during deployment, be expanded to occlude the perforating vein 11, before the junction with the deep vein 12. For example, perforating vein ablation apparatus 10 may be deployed approximately 1 cm from the deep vein, or other suitable distances so as to allow treatment to be implemented, yet to ensure that the apparatus is safely deployed and anchored before the entry into the deep vein. Positioning of the apparatus may typically be done in real time, for example, using ultrasound, X-Ray, or other suitable imaging.

Perforating vein ablation apparatus 10 may also include an anchoring mechanism 14, for example, a hook, anchor, pin etc. to anchor itself into the perforating vein wall(s), to prevent movement of the perforating vein ablation apparatus 10 after deployment. As can be seen in FIGS. 1B and 1C respectively, anchoring mechanism 14 may include 1 or more anchors or hooks. Anchors or hooks may be attached to the distal and/or proximal ends of vein ablation apparatus 10. In other embodiments anchors or hooks may be placed in any selected position(s) on vein ablation apparatus 10, to enable effective anchoring into the target vein. Perforating vein ablation apparatus 10 may further include an initial positioning fixing mechanism(s) 16 and 17, for example, small hooks, pins or thorns, to enable initial sealing of the perforating vein ablation apparatus 10 position when initially deployed. Verification that the Perforating vein ablation apparatus 10 has been accurately deployed may be achieved by viewing the apparatus using ultrasound or X-ray imaging. For example, if the anchor 14 can be viewed as having penetrated the vein wall, thereby indicating that the apparatus has been successfully deployed and anchored in the vein. Other materials or combinations of materials may be used.

Perforating vein ablation apparatus 10 may be positioned in a perforating vein 11, for example, using syringe driver or other suitable guidance mechanism. The guidance may take place using ultrasound to help locate and guide the device, or using other suitable guidance technologies. According to some embodiments, the syringe driver may guide device 10 directly from outside the limb, for example, from the outer side of the leg, in the perforant area. Other access positions may be used, where appropriate, to access a target vein. A syringe with a pre-loaded needle may be used to deploy perforating vein ablation apparatus 10, to enable fast and accurate reaching of the target area, and pushing the syringe plunger to release the perforating vein ablation apparatus 10. The syringe plunger may be retracted to retract perforating vein ablation apparatus 10. In other embodiments the syringe plunger may be retracted to aspirate blood or debris before, during or following deployment of the ablation apparatus. In other embodiments the syringe plunger may be retracted to manipulate the positioning of the vein ablation apparatus 10, to help with its deployment or release.

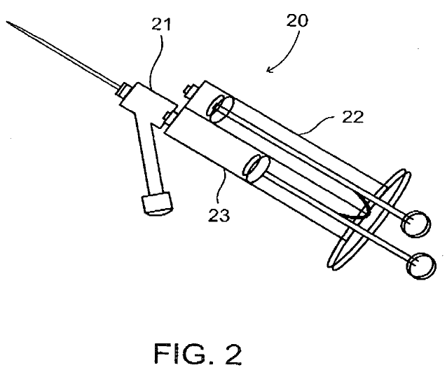

Reference is now made to FIG. 2, which is a schematic drawing showing a syringe driver, according to some embodiments. As can be seen in FIG. 2, the syringe driver 20 may be pre-loaded with a second needle or lumen, which may be controlled using a second needle plunger or applicator 22. When the occluding apparatus reaches the selected target area, the needle may be used to aspirate blood, debris or other materials, for example to reduce pressure in the vein, reduce the vein size, and/or verify the occluding apparatus's position. The procedure may be performed under ultrasonic or other guidance, and may be performed before, during or after deployment of the perforating vein ablation apparatus 10. According to some embodiments of the present invention, the needle of the second plunger 22 may be pre-loaded with a ligation or treatment element (e.g. sclerosing agents), which may be discharged into the vessel, using the syringe plunger.

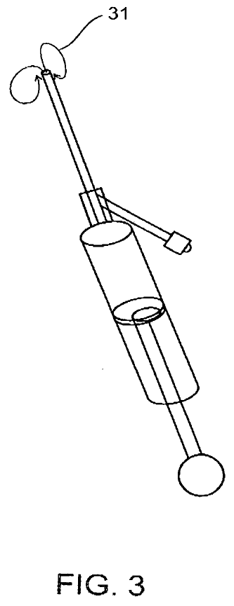

Reference is now made to FIG. 3, which is a schematic drawing showing a perforating vein ablation apparatus 30 with expandable suturing capacity, according to some embodiments. As can be seen in FIG. 3, Perforating vein ablation apparatus 30 may include one or more expandable threads 31 (e.g., Nitinol) coupled to surgical suture. The threads may expand upon deployment in a perforating vein to close, tie, stitch or otherwise ligate the vein, hereinafter referred to as perforant inner ligation. Other locations may be used for application of the above treatment. In some embodiments a dual syringe driver may be used to aspirate blood or debris from a target vein, either before, during or after the perforating vein ablation apparatus deployment.

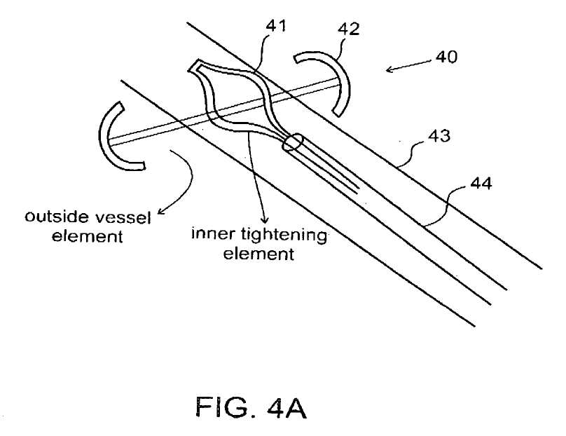

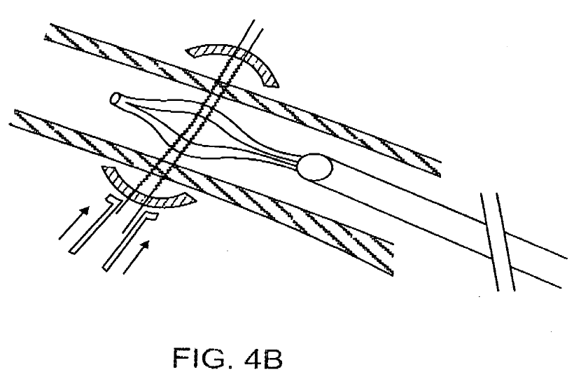

Reference is now made to FIG. 4A, which is a schematic drawing showing a perforating vein ablation apparatus 40 with a vein catching mechanism, according to some embodiments. As can be seen in FIG. 4A vein catching mechanism 40 may be entered into a vein through needle or lumen 44, and following deployment, it may be expanded into a position where it may ligate, ablate, occlude or destroy the vein. Following the entry of vein catching mechanism 40 into the target vein, a vein locking mechanism may be entered from an external position adjacent to the vein being caught. This mechanism may be entered using a catheter or syringe element, for example, and may enter locking braces 42 around the target vein, to help anchor the vein catching mechanism to the vein wall. Vein catching mechanism may thereby be anchored into or through the walls of the vein, to maintain its position and prevent forward or backward movement in the vein. In some embodiments the catching mechanism may be naturally or automatically expandable, for example, it may be expanded and controlled using a syringe guide, plunger, string, needle applicator, or catheter controller. In one embodiment the catching mechanism may be constructed from expandable materials such that upon deployment it may instinctually move into a desired position to achieve the vein ligation. In some embodiments the vein catching mechanism may be constructed from medical grade plastics, and may act for example as a ratchet mechanism to hold the vein. Other materials and configurations may be used. Additionally, aspiration of blood may be performed before, during or following the deployment of the catching mechanism.

Reference is now made to FIG. 4B, which is a schematic drawing showing the penetration of vein catching mechanism into the vein, according to some embodiments. As can be seen in FIG. 4B, in some embodiments the vein catching mechanism may be entered into the target vein using a needle or catheter, where the inner catching braces may be deployed, to pressure the vein catching mechanism towards the vein walls, from inside the vein. Following the entry of vein catching mechanism into the target vein, a vein locking mechanism may be entered from an external position adjacent to the vein being caught. This mechanism may be entered using a catheter or syringe element, for example, and may enter locking braces around the target vein, to help anchor the vein catching mechanism to the vein wall. As can be seen in the figure, a ratchet type mechanism may be used, wherein the external braces may be pushed together to eventually lock onto each other, thereby strangling the vein. In some embodiments aspiration of blood may be performed before, during or following the deployment of the catching mechanism. Of course, other structures and dimensions may be used. Additionally, any combination of the above steps may be implemented. Further, other steps or series of steps may be used.

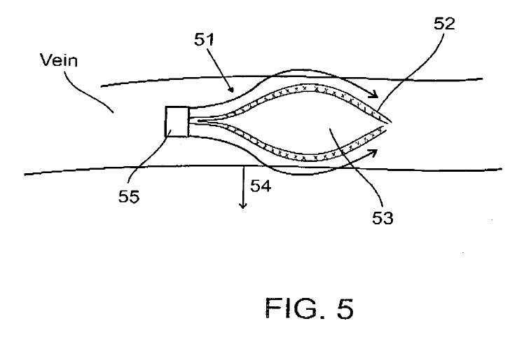

Reference is now made to FIG. 5, which is a schematic drawing showing a vessel ablation apparatus and method, according to some embodiments. As can be seen in FIG. 5, the ablation apparatus 51 may include a netting based device 52 that surrounds an expandable balloon 53, the device being deliverable via a catheter. The ablation device may further include one or more needles or anchors 54, for example, with hooks or spikes for anchoring the device in a target vein. Anchors 54 may be positioned in the base 55 of ablation apparatus 51, and may be deployed by releasing them, for example, pulling or pushing them beyond base 55. Anchors may be constructed from Nitinol, stainless steel, or other memory alloys or metals.

According to some embodiments, the netting device 52 may be entered into a target vessel. When in position the balloon may be expanded, for example, by entering liquid, bubbled liquid (that may be viewed via ultra sound) etc. to expand the balloon, thereby expanding netting towards the vessel wall. Next, the anchors may be released or deployed, thereby forcing the anchors around the netting, until the anchors enter or penetrate the vessel walls, thereby anchoring the device to the vessel walls. Subsequently, the balloon may be deflated thereby shrinking the ablation apparatus, and also causing the anchored vein walls to be collapsed or brought close together, thereby closing or narrowing the target vessel. The netting used may subsequently be removed together with the balloon, or one or more of these elements may be left in the vessel. In some embodiments the netting may be biodegradable and may dissolve in the vessel.

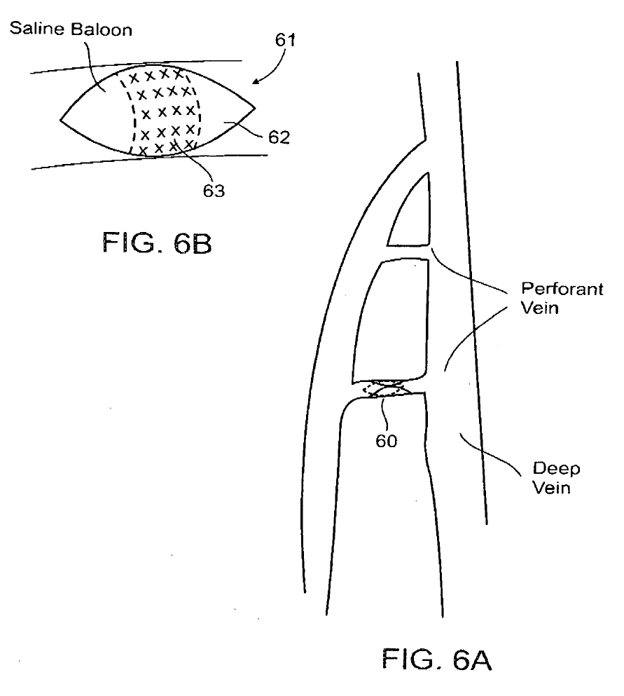

Reference is now made to FIGS. 6A and 6B, which are schematic drawings showing an apparatus and method for ablating a target perforant vessel, according to some embodiments. As can be seen in FIG. 6A, the ablation apparatus may be deployed in the perforant vein, prior to the perforant junction where the perforant vein meets the deep vein. As can further be seen in FIG. 6B, the ablation apparatus 61 may include an expandable balloon 62 surrounded by a mesh or netting structure 63. In some embodiments mesh structure 63 may be limited substantially to the area of greatest diameter, or the widest part, of the inflated balloon, which is most likely to make contact with the vein wall. Netting structure 63 may include netting that includes loose ends up to approximately 5 mm that when expanded, will act like thorns, pins or hooks, to expand into the vessel wall, and thereby anchor themselves in the wall. For example, the netting with external hooks may be positioned around the mid-center of the expandable balloon. When the balloon is subsequently deflated the vessel may substantially collapse, thereby closing or collapsing the anchored hooks, thereby substantially closing the vessel. In some embodiments, pins or edges required for anchoring into the vein wall may be longer or shorter than 5 mm. In some embodiments the mesh may be constructed from Nitinol or other suitable shape alloys, for example, that may be pre-configured to expand, constrict, and “click” itself into anchoring position.

According to some embodiments, a catheter may deliver the balloon into the perforant vein, for example, under ultrasound or other suitable guidance systems. When the balloon is in position, for example in the vein-valve area prior to the perforant junction, it may inflated, for example using saline or other suitable inflation means. The inflation of the balloon causes the mesh surrounding at least parts of the balloon to expand accordingly, such that the angles of the mesh parts, ends or hooks extend substantially outwards towards the vein wall, to hook into or anchor into the vein wall. In some embodiments the mesh may be pre-configured, for example, using shape materials, to deploy at an angle appropriate for anchoring into a vein wall. The balloon may be subsequently deflated, causing a consequent constriction of the mesh. Since the mesh is substantially hooked to the vein wall, the constriction of the mesh may cause the sides of the vein wall to be brought together, thereby substantially closing or narrowing the vein. In some embodiments the hooks may further catch each other following the constriction of the mesh, thereby “locking” the vein walls together, using the mesh. In some embodiments the mesh may be controlled to function as a result of the inflation and deflation of the balloon. In other embodiments the mesh may be constructed from shape alloys that are configured to deploy, expand, constrict and lock etc., in suitable circumstances.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A vein ablation apparatus for implementing perforating vein treatment, comprising:
a vein ablation blocking element, to be deployed downstream from said vein ablation apparatus, before a deep vein junction, to prevent passage to a downstream deep vein; and
an anchoring mechanism, to anchor said vein ablation apparatus into the perforating vein wall(s), to prevent movement of said vein ablation apparatus after deployment.

2. The vein ablation apparatus of claim 1, wherein said blocking element is a controllable mesh element designed to block off an area of greatest diameter in a target vein.

3 The vein ablation apparatus of claim 1, wherein said anchoring mechanism includes hooks designed to catch each other following the constriction of said anchoring apparatus, thereby locking the vein walls together.

4. The vein ablation apparatus of claim 1, wherein said anchoring mechanism is pre-configured to deploy at an angle appropriate for anchoring into said vein wall.

5. The vein ablation apparatus of claim 1, wherein said anchoring mechanism may include one or more anchoring elements selected from the set consisting of hooks, anchors, pins, and latches.

6. The vein ablation apparatus of claim 2, wherein said anchoring mechanism is attached to the distal and/or proximal ends of said vein ablation apparatus.

7. The vein ablation apparatus of claim 2, wherein said anchoring mechanism includes an initial positioning fixing mechanism to enable initial sealing of the perforating vein ablation apparatus position when initially deployed.

8. The vein ablation apparatus of claim 2, wherein said vein ablation apparatus includes one or more expandable threads coupled to surgical suture.

9. The vein ablation apparatus of claim 1, comprising an external vein locking mechanism that is deployable from an external position adjacent to a target vein, to help anchor said anchoring mechanism to a vein wall.

10. A method for enabling perforating vein treatment, comprising:
entering a netting device into a target vessel using an introducing catheter;
when said netting device is in position, expanding a balloon to expand said netting towards a target vessel wall;
releasing one or more anchors, thereby forcing said anchors around said netting, until said anchors penetrate said vessel walls, thereby anchoring the device to said vessel walls; and
deflating said balloon, thereby shrinking the ablation apparatus, and causing said anchored vein walls to be collapsed.

11. The method of claim 10, comprising removing said netting together with said balloon.

12. The method of claim 10, wherein a syringe driver may guide said ablation device directly from outside the limb.

13. The method of claim 12, wherein a syringe driver syringe with a pre-loaded needle may be used to deploy said vein ablation device.

14. The method of claim 12, comprising retracting said syringe plunger to aspirate blood or debris before, during or following deployment of said ablation device.

15. The method of claim 12, comprising retracting said syringe plunger to manipulate the positioning of said vein ablation device.

16. A perforant vein ablation device comprising:
a netting based device that surrounds an expandable balloon, the device being deliverable via a catheter;
one or more anchors for anchoring the device in a target vein, said anchors being positionable at the base of the ablation device, and said anchors being deployable by releasing them beyond said base.

17. The device of claim 16, wherein said anchors are constructed from one or more materials selected from the group consisting of Nitinol, stainless steel, or other memory alloys or metals.
